(19) United States
(12) Patent Application Publication
(10) Pub. No.: US 2012/0064015 A1
(43) Pub. Date: Mar. 15, 2012

(54) Food Additive Composition as an Agent for Reducing the Adhesion of Dental Biofilms in Sweetened Products

(76) Inventor: Dominique Valeri, Paris (FR)

(21) Appl. No.: 13/148,596

(22) PCT Filed: Feb. 9, 2010
(86) PCT No.: PCT/EP2010/051533

§ 371 Date: Sep. 9, 2011

(30) Foreign Application Priority Data
Feb. 10, 2009 (FR) 09/50822
Jul. 30, 2009 (FR) 09/55366

(51) Int. Cl. A61K 36/537; A61P 31/04; A61K 9/68

(52) U.S. Cl. 424/48; 424/746

(57) ABSTRACT

The present invention relates to a food additive composition to be added to sugar or sweetened products containing cariogenic free sugars or by-products thereof, enabling a reduction of the adhesion of dental biofilms and the appearance of caries while strengthening the tooth and the dental support tissue. The present invention also relates to food products made of cariogenic free sugars or by-products thereof containing said food additive product, as well as to a method of manufacturing such a food product containing said food additive composition, such as a chocolate.

The present invention concerns a food additive composition intended to be incorporated in sugar or sweetened products containing cariogenic free sugars or derivatives thereof, reducing the adhesion of dental biofilm and the appearance of caries while strengthening the teeth and the dental support tissue. The present invention also concerns food products based on cariogenic free sugars or derivatives thereof containing this food additive composition and a method of manufacturing such a food product containing this food additive composition such as a chocolate.

Tooth decay is the third scourge in the world after cancer and cardiovascular diseases. According to data from the World Health Organisation, 60% to 90% of children of school age in industrialised countries have caries, as well as a large majority of adults. Tooth decay is also the most usual mouth ailment in several countries in Asia and Latin America.

Moreover, periodontal diseases, such as gingivitis, affect 80% of European adults and 50% of adolescents aged 15.

There is a direct link between sugar consumption and caries.

In order to colonise dental tissues, bacteria organise themselves by establishing a strong relationship of adhesion on the enamel of the teeth but also with each other. If this process is not interfered with, in particular by suitable brushing, a yellowish deposit forms very quickly and visibly: dental plaque or biofilm. This biofilm is a complex set of various bacterial populations encompassed in a matrix of polysaccharides and glycoproteins that reduces access of salivary buffers to the dental surfaces proportionally to its development. The bacteria in dental plaque ferments monosaccharides (e.g. fructose, glucose and galactose), disaccharides (e.g. saccharose, maltose and lactose) and polysaccharides (e.g. starch) taken in with food. During this process, acids form as close to the teeth as possible, thus causing repeated lowerings of the pH causing solubilisation of the crystals of the hydroxyapatite forming the enamel on the teeth and in the long term a demineralisation of the teeth. For a caries to develop, four conditions must be combined:

the presence of bacteria in the plaque on the surface of the tooth (mainly Streptococcus mutans (S. mutans));

availability of fermentable sugars (especially saccharose);

a more or less fragile host (thickness of the enamel, teeth badly positioned, etc.);

sufficient duration (the adhering products are therefore more cariogenic).

The bacteria in the plaque also act on the periodontal tissues, releasing irritant products (urea, ammonia, etc.), enzymes and toxins, which trigger an inflammatory and immunological reaction of the gums, thus causing gingivitis. Elimination of the anti-infectious agents may lead to an overproduction of cytokines which, instead of being protective, become destructive of the periodontal structures and thus lead to periodontitis, i.e. definitive recession of the supporting tissue, which in extreme circumstances may lead to loosening and loss of teeth.

Sugars are thus the main food factor associated with caries and more generally with periodontal diseases.

Though fluorine fulfils an unquestionable role in the prevention of caries, it nevertheless does not eliminate these pathologies and many communities are not exposed to optimum quantities of fluorine. Moreover, the dosage of fluorine that affords an effect of strengthening teeth by transforming the hydroxyapatite of the enamel into fluoroapatite is not unanimously accepted because of the risks of secondary pathologies incurred (fluorosis, neurological consequences, etc.).

The food industry moreover offers many products in which sugar is replaced by sweeteners that have little or no cariogenicity while having a high sweetening capacity.

However, the sweeteners used in the food industry are often synthetic products and, although use thereof has been validated by the regulating authorities, they worry populations who prefer to avoid such chemical products and the carcinogenic image thereof in favour of products of natural origin.

In order to prevent the development of periodontal diseases and in particular caries, the World Health Organisation recommends above all reducing the quantity of free sugars, as well as a food regime rich in food value, fruits and vegetables containing starch and fibres, which appear to be associated with a low level of caries, and containing little free sugar and fat.

Another alternative appeared during the 1970s with the use of xylitol for the first time in a dental application (study by Turku). Xylitol is a natural sugar alcohol with five carbon atoms isolated from beech chips by the chemist Fischer at the end of the 19^{th} century. This has interesting properties:

it inhibits the growth of the bacteria responsible for dental plaque, which are incapable of metabolising and therefore do not produce cariogenic acids;

particularly in gum form, it stimulates the secretion of saliva and therefore promotes this natural defence system against caries (buffer effect increased, production of amylase and peroxydase, etc.);

it is less calorific than saccharose for an equal sweetening capacity.

Although its efficacy no longer needs to be proved, the use of xylitol is still a matter of debate in terms of cost and accessibility to all. This is because the cost of commercial production thereof, which is carried out using birch, maize and sugar cane by hydrolysis and hydrogenation of xylane, remains high. Moreover, the effects of the anti-cariogenic efficacy thereof as such are measured on the regularity with which it is taken in the long term. Though changes in the buccal bacterial flora are observed within 24 hours following taking xylitol, it is considered that 2 to 3 years of regular taking in order to guarantee residual durable anti-caries protection is necessary. Such regularity entails intentional action over a long period and the establishment of a monitoring and training system in a prevention programme, which represents a not insignificant cost.

In addition, although having a sweetening capacity identical to that of saccharose, xylitol has a marked mentholated taste, which reduces the possibilities of use thereof in high doses, in replacement for saccharose in the majority of sweetened food products.

Moreover, daily consumption thereof should not exceed 10 g for an adult, according to the list of excipients with a recognised effect that appeared in the Official Journal of 12 Oct. 2007, a quantity beyond which the risks of osmotic diarrhoea are high. General belief does however very often place this threshold at 20 to 30 g for an adult.

In 2008, the European Food Safety Authority issued an opinion on the scientific justification of a health allegation concerning chewing gums/pastilles with xylitol and a reduction in the risk of dental caries. This allegation made reference to sweetened chewing gums containing 100% xylitol and sweetened pastilles with at least 56% xylitol. It was brought out that only the chewing gums with xylitol reduce the risk of dental caries in children, this effect being related to a daily consumption of 2-3 g of sweetened chewing gum at least three times a day after meals.

Current use of xylitol is therefore associated with its anti-cariogenic effect as such, i.e. as a substitute for conventional (cariogenic) sugars that cannot be metabolised by the bacteria of the dental plaque and therefore be at the origin of the production of acids responsible for the initiation of the caries process.

Use thereof as a substitute for conventional sugars is however limited by the maximum daily ingestion dose equal to 10 g whereas the daily consumption of free sugars recommended by the WHO is 62 to 94 g, a consumption that may as much as triple in “over-fed” countries (220 g per adult per day in Canada).

The use thereof as a substitute for conventional sugars is also limited by its unsuitable mentholated taste in many products.

Moreover, the consumption of xylitol in the form of chewing gums, a formulation deemed to reinforce the action thereof by the associated mastication effect, has numerous limits:

it must be avoided for children aged less than three years given the high risk of choking within this age group;

it can not be easily controlled in terms of quantity and may exceed the limit dose advised, causing risk of osmotic diarrhoea;

it may give rise to gastro-oesophageal and intestinal pathologies;

the procedure must be intentional;

the consumption of such products is not easily accessible to all since it is expensive;

access to xylitol by this means takes place in association with numerous additives, dyes, moisture-keeping agents (E414, E222, E271, etc.).

The present invention concerns a food additive composition that remedies all these drawbacks. It contains the elements that reduce the appearance of caries as well as the elements that strengthen the tooth and the dental support tissues. This composition is remarkable in a first aspect in that it has a synergy between an agent reducing the adhesion of the dental biofilm, a remineralising agent and an antiseptic agent. This composition is remarkable in a second aspect in that it is intended to be incorporated in sweetened products containing cariogenic free sugars or derivatives thereof, thus reducing the appearance of caries while strengthening the tooth and the dental support tissues, without any change in food habits.

The subject matter of the present invention is thus a food additive composition comprising: a) an agent reducing the adhesion of dental biofilm, b) a mineralising agent based on silicon and/or based on calcium, c) an antiseptic.

“Food additive composition” means a composition intended to be added to a food product, that is to say a product that is consumed by living beings (human or animal) for energy or nutritional purposes.

According to the present invention, “agent reducing the adhesion of the dental biofilm” means an agent that is capable of reducing the number of S. mutans fixed to the tooth surface and/or cohesion between these different streptococci and/or the cohesion of the polysaccharides forming this biofilm and which therefore limits the adhesion and/or development of the biofilm or dental plaque. The expression “agent reducing the adhesion of the dental biofilm” also includes a possible mixture of several agents described in the corresponding list below, which may individually fulfil the function of “agent reducing the adhesion of dental biofilm”. The anti-adhesion agent according to the present invention acts by interfering with the metabolism of the bacteria that make up the biofilm, in particular by interfering with their process of adhesion and/or colonisation on the tooth surfaces or periodontal tissues. There exist many methods for identifying such agents for reducing the adhesion of dental biofilm. For example, tests for assessing the adhesion of Streptococcus mutans on solid bases or tissues or making it possible to note a reduction in dental plaque on hydroxyapatite are described Bertrand et al (Les cahiers de l'ADF, No. 14-15, 2^{nd} quarter 2003), Engels-Deutsch et al. (Les cahiers de l'ADF, No. 16-17, 3^{rd} quarter 2003) or WO 97/38670.

According to the present invention, “mineralising agent” means an agent that is capable of providing dental organic tissues, in particular to teeth, enamel, dental bone and periodontal tissues, preferably to the dental bone, with mineral substances in a form that can be assimilated by said tissues. The mineralising agent according to the invention contains at least silicon and/or calcium, or at least one of the derivatives thereof, in particular organic, such as silica (SiO₂). The expression “mineralising agent” also includes a possible mixture of several agents described in the corresponding list below, which may individually fulfil the function of “mineralising agent”.

According to the present invention, “antiseptic” means any molecule or extract containing such molecule that destroys the infectious agents such as bacteria for example, or opposes the proliferation thereof. In periodontics, such agents are for example non-limitatively chlorhexidine, hexetidine, phenols, quaternary ammoniums, oxidizing agents and plant extracts. The expression “antiseptic” also includes a possible mixture of several agents described in the corresponding list below, able to exert individually the function of “antiseptic”.

In a preferred embodiment, the composition according to the present invention consists entirely of ingredients of natural origin.

In this preferred embodiment, the agent reducing the adhesion of dental biofilm of the composition according to the present invention can in particular be chosen from polyols, cranberry, coffee, chicory, tea or grapes, in particular raisins or red wine. The polyols serving as an agent reducing the adhesion of dental biofilm can in particular be chosen from:

hydrogenated monosaccharides, such as xylitol, sorbitol, mannitol or erythritol;

hydrogenated disaccharides, such as maltitol, lactitol, hydrogenated isomaltulose (an equimolar mixture of glucopyranosyl-1,6-sorbitol and glucopyranosyl-1,1-mannitol);

inulin.

The agent reducing the adhesion of dental biofilm is preferably xylitol, which can be extracted from birch bark for example. As previously stated, several of these agents reducing the adhesion of biofilm can be used in the composition according to the present invention.

In this preferred embodiment, the mineralising agent based on silicon of the composition according to the present invention is advantageously a substance of plant origin containing silica extracted from plants including horsetail (Equisetum arvense containing about 80% silica in its stem and 60% silica in the entire plant), lithothamnion, alfalfa, soya, nettle, bamboo or meadowsweet. These plants can form part of the composition of the present invention directly as a mineralising agent adding silica, in the form of a dried powder of homogenates of the entire plant concerned or a part thereof. Likewise, this substance of plant origin may first of all be obtained in liquid form from a homogenate of the entire plant or a part thereof and secondly dried to obtain a powder. Various methods of extraction (hydrodistillation, solvent extraction, ultrasonic extraction, pressing, decoction, maceration, infusion, pressing, cryogrinding, enfleurage), filtration (ultrafiltration, nanofiltration) and purification such as chromatography can be used to concentrate the homogenate in liquid form as a mineralising agent before drying it or lyophilising it in order to obtain a powder. Preferably, this mineralising agent based on silica is horsetail. As stated previously, several of these mineralising agents can be used in the composition according to the present invention.

In this preferred embodiment, the antiseptic or antimicrobial agent of the composition according to the present invention is advantageously a substance extracted from plants including sage (Salvia officinalis), echinacea, myrrh, propolis, ratanhia peruviana, calendula, yam, camomile, aloe vera, acerola, sanguinarine, lemon or grapefruit. These plants can form part of the composition of the present invention directly as an antiseptic adding silica, in the form of a dried powder of homogenates of the entire plant concerned or part thereof. Likewise, this substance of plant origin may first of all be obtained in liquid form from a homogenate of the entire plant or a part thereof and secondly dried to obtain a powder. Various methods of extraction (hydrodistillation, solvent extraction, ultrasonic extraction, pressing, decoction, maceration, infusion, pressing, cryogrinding, enfleurage), filtration (ultrafiltration, nanofiltration) and purification such as chromatography can be used to concentrate the homogenate in liquid form as a mineralising agent before drying it or lyophilising it in order to obtain a powder. Preferably, the composition according to the invention contains sage as an antiseptic. As previously stated, several of these antiseptics can be used in the composition according to the present invention.

Thus, in this preferred embodiment, the composition according to the present invention is characterised in that:

a) the agent for reducing the adhesion of dental biofilm is chosen from the group consisting of:

polyols such as xylitol, sorbitol, mannitol and erythritol, and hydrogenated disaccharides such as maltitol, lactitol and hydrogenated isomaltulose,

cranberry, coffee, chicory, inulin, tea, grapes, raisins and red wine,

b) the mineralising agent based on silicon is chosen from the group consisting of:

silica,

the substances of plant origin extracted from plants including horsetail, lithothamnion, alfalfa, soya, nettle, bamboo or meadowsweet,

c) the antiseptic is a substance extracted from plants chosen from sage, echinacea, myrrh, propolis, ratanhia peruviana, calendula, yam, camomile, aloe vera, acerola, sanguinarine, lemon or grapefruit.

The composition according to the present invention may also contain in addition an agent strengthening the dental support tissues, preferably an oil in particular chosen from vegetable oil such as avocado oil, evening primrose oil, borage oil or soya oil, or from animal oils containing omega 3 or 6, in particular fatty fish oils. Preferably, the composition according to the invention contains avocado oil as an agent strengthening the dental support tissues. By way of information avocado oil contains approximately 42% to 63% oleic acid, 17% to 29% palmitic acid, 9% to 16% linoleic acid, less than 1% linoleic acid; 1% to 2% unsaponifiable consisting half of branched hydrocarbons with 20% unidentified reducing sterols and triols.

In this case, the composition according to the present invention is characterised in that the composition also contains an agent strengthening the dental support tissues chosen from vegetable oils or avocado, evening primrose, borage or soya, or from animal oils of fatty fish containing omega 3 or 6.

Particularly preferably, the composition according to the present invention is characterised in that it contains xylitol as an agent reducing the adhesion of dental biofilm, horsetail as a silica-based mineralising agent and sage as an antiseptic.

In addition, the composition according to the present invention is characterised in that it may also contain avocado oil as an agent strengthening the tooth support tissues.

In a first aspect, the composition according to the present invention is remarkable in that it has a synergy between an agent for reducing the adhesion of dental biofilm, a remineralising agent and an antiseptic.

The bacteria responsible for dental pathologies (S. mutans) use 6-carbon sugars as a primary energy source. The Streptococci mutans in the mouth cavity colonise the tooth surfaces by means of adhesions, which are protein constituents of the bacterial wall. S. mutans fulfils a key role both in the triggering and the development of the caries process thanks to its metabolism of the homofermentary type by degrading the food sugars and producing acids, but also its ability to produce polysaccharides that participate in the adhesion of the bacteria (to the tooth surfaces and to each other) in order to form dental plaque or dental biofilm.

Studies show that, at very low doses, xylitol interferes with the metabolism of S. mutans in the mouth. Xylitol is transported inside the S. mutans cells via the phosphotransferase system of fructose, phosphorylated into xylitol 5-phosphate, which cannot be fermented or metabolised and accumulates in the intracell vacuoles causing rapid inhibition of the growth of S. mutans. In addition, a futile xylitol cycle may be set up, also inhibiting the bacterial growth indirectly, by successive phosphorylation and dephosphorylation of the xylitol for respectively its entry and expulsion from the cells. However, S. mutans adapt quickly (about 24 hours) and become resistant to the xylitol (they are called S. mutans XR). During this adaptation, the S. mutans XR modify some of their metabolic pathways, thus enabling the xylitol to enter cells via a permease and allowing its non-phosphorylisation into xylitol 5-phosphate, thus preventing triggering of the futile cycle and any inhibiting effect on the growth of the bacteria. These adaptations cause in S. mutans XR an alteration in the capacity to synthesise the extra- and intra-cellular polysaccharides that form the framework of the dental biofilm. Consequently this biofilm becomes less adhesive to the tooth surfaces. Since S. mutans XR have a faster reproduction ability than the parent strains, they therefore become the majority within the biofilm and thus make it progressively less adhesive and less virulent.

In the context of the present invention, polyols, and in particular xylitol, are used as agents for reducing the adhesion of biofilm rather than as non-cariogenic sweeteners.

Surprisingly, in the context of the composition of the present invention, the combination of xylitol with a mineralising agent and an antiseptic as defined above creates a synergy increasing the beneficial effects of these components in combating the caries process. Without wishing to be limited to a particular theory, it is possible that the aforementioned destabilisation of biofilm by the adhesion-reducing agent facilitates access of the mineralising agent and antiseptic agent to the dental surface and to the periodontal tissues, which potentialises the beneficial actions thereof. This additional effect afforded by the composition according to the present invention is therefore not a simple addition of the effects of the various components but a combination of effects limiting the appearance of caries while strengthening the teeth and dental support tissues by destabilising the adhesion of dental biofilm.

The second aspect according to which the composition according to the present invention is remarkable is that it is intended to be incorporated in sugar products containing cariogenic free sugars or derivatives thereof, reducing the appearance of caries while strengthening the teeth and dental support tissues, without any change in food habits.

Cariogenic free sugars or derivatives thereof means all monosaccharides (simple sugars) and disaccharides (sugars composed of two monosaccharides), including refined cane, beet and maize sugars, such as saccharose, glucose and fructose for example, but also derivatives thereof, present naturally in certain foods (honey, syrups, fruit juices) or added, trigging the caries process. Sugars similar to the above in the oses family (aldoses and ketoses), such as anomers and epimers thereof for example, which may trigger the caries process, are also included in this composition; as well as derivatives of the above sugars such as polyols derived from oses by reduction of the aldehyde or ketone group of a carbohydrate.

Entirely surprisingly, the synergy described above between the xylitol used as an agent reducing the adhesion of dental biofilm, a remineralising agent and an antiseptic in the context of the composition according to the present invention, also operates when the composition of the present invention is added to products with a high proportion of cariogenic free sugars or derivatives thereof. The composition according to the present invention reduces the cariogenic effect of the sugar product in which it is incorporated while exerting a beneficial strengthening action around the tooth and dental support tissues such as the gums and the dental bone.

The composition according to the present invention is intended to be incorporated in sugar products. It reduces the cariogenic effect of the sugar product in which it is incorporated while exerting a beneficial strengthening action around the gums and the dental bone.

The composition according to the present invention thus overcomes a commonly accepted prejudice by reducing the caries process despite the absorption of cariogenic free sugars or derivatives thereof by reducing the composition and adhesion of dental plaque despite this absorption of cariogenic free sugars or derivatives thereof.

Surprisingly, the composition according to the present invention makes it possible to disregard a possibly effect of competition between the polyols, in particular the xylitol, and the cariogenic free sugars or derivatives thereof, present in the food products to which the composition according to the present invention is added; even in the cases where the cariogenic free sugars or derivatives thereof are in a great majority in the food products to which the composition according to the present invention is added.

By a synergic action, the association of the polyols and in particular xylitol, as an agent for reducing the adhesion of dental biofilm, with a remineralising agent and an antiseptic, potentialises the reduction in cohesion and adhesion of dental plaque, the remineralising capacity and the cleaning of the teeth, gums and dental bone.

The effect of reducing the adhesion of biofilm by polyols and in particular xylitol, as well as the synergic action of the different components, is observable and therefore usable for a low final percentage of the different components of the food additive composition according to the present invention, added to a food product. In particular, the quantity of xylitol present in the food additive composition according to the present invention is such that the final percentage thereof in a food product containing mainly cariogenic free sugars or derivatives thereof does not exceed 0.8% (w/w).

Compared with the solutions proposed in the prior art, the food additive product that is the subject matter of the present invention therefore has the following major advantages:

it is easy to manufacture and inexpensive;

it can be entirely manufactured from natural elements of plant origin;

the food product to which it is added keeps its normal sugar content;

it does not cause any change to the sweetened flavour or texture of the food product to which it is added;

it does not have the mentholated taste particular to the use of a certain proportion of xylitol;

it does not have any risk of osmotic diarrhoea through the low take-up of xylitol that it entails;

it does not present any risks of gastro-oesophageal and intestinal pathologies related to the consumption of chewing gum.

Equally, this food additive composition:

suits all age categories, including the youngest;

does not change food habits highly oriented physiologically, psychologically and socially towards the intake of sugar;

does not require any intentional procedure in order to be assimilated since it is incorporated in highly usual food products;

guarantees a regular take-up of xylitol;

is accessible to all in terms of cost.

Moreover, the food additive composition affords protection proportional to the take-up of sugar, quantitatively and qualitatively. This is because the protection exerted by the components of the composition of the present invention will be all the higher, especially since the take up of sugar containing this composition according to the present invention is repeated, large and with high cariogenic capability (sticky sugar in confectionery). The more regular and spread over time the take-up of sugar containing the composition of the present invention, the more the sugar will be adhesive and the more the teeth will then be in contact with the beneficial components of the present food additive composition.

The food additive composition according to the present invention therefore, without changing the food habits of the populations that, from birth, have physiologically and psychologically a pronounced taste for sugar and all products containing it, reduces the caries process by destabilisation of the dental biofilm, while strengthening the teeth and the dental support tissues. This composition therefore makes it possible, through the addition thereof, to normal sweetened products, to profit from established food habits and to take advantage of the destructive aspects of sugar to perform actions that curb the caries process and are reparative.

The composition that is the subject matter of the present invention comprises in particular:

a. about 79 to 99 parts by weight, preferably 90 to 96 parts by weight of xylitol as an agent for reducing the adhesion of dental biofilm;

b. about 0.5 to 19 parts by weight, preferably 2.5 to 10 parts by weight of silica or a silica source of plant origin such as horsetail as a remineralising agent;

c. about 0.2 to 2 parts by weight, preferably 0.4 to 1 part by weight of an antiseptic of plant origin such as sage.

The composition that is the subject matter of the present invention may also comprise about 0.2 to 2 parts by weight, preferably 0.4 to 1 part by weight of an agent strengthening the dental support tissues.

Part by weight means the proportion of the mass of a component with respect to the sum of the masses of the components, multiplied by 100, in other words the percentage by mass of a component in the total composition.

As mentioned previously, the composition according to the present invention is intended to be incorporated in sugar products. In this regard, it can be used as an agent reducing the cariogenic capability of a food product containing free sugars and also as an agent for strengthening the dental support tissues.

As a food additive or food additive composition, the composition according to the present invention preferably contains at least 79% by weight, preferably at least 85% by weight, even more preferentially at least 95% by weight of an agent for reducing the adhesion of dental biofilm, preferably at least 0.5% by weight, preferably at least 2.5% by weight, even more preferentially at least 10% of a mineralising agent based on silicon and at least 0.2% by weight, preferably 0.4% by weight, even more preferentially 1% of an antiseptic.

It can be used in solid form, for example in the form of powder or granules, or in liquid form in association with a suitable liquid carrier. It can also be used in the form of gel. It can be incorporated directly in an already sweetened food product or in the sugar itself, prior to incorporation thereof in the food product that it is wished to sweeten. It may be in one of the forms previously cited so as to be added extemporaneously, by the consumer in a product of his choice.

For example, the composition according to the invention can advantageously be added to cane sugar or beet sugar that contains saccharose, to milk sugar that contains lactose, to malt sugar that contains maltose, to fruit sugar that contains fructose or to grape sugar that contains dextrose and glucose.

For use thereof as an agent for reducing adhesion of dental biofilm in a food product containing cariogenic free sugars or derivatives thereof, the food additive composition according to the present invention adds to said food product:

a. from 0.1% to 0.8% by weight, preferably 0.3% to 0.6% by weight xylitol as an agent for reducing the adhesion of dental biofilm;

b. from 0.005% to 0.02% by weight, preferably 0.01% to 0.02% by weight silica or a source of silica of plant origin, such as horsetail, as a remineralising agent;

c. from 0.001% to 0.01% by weight, preferably 0.002% to 0.003% by weight of an antiseptic of plant origin such as sage.

For use more specifically oriented towards strengthening of the dental support tissues, the food additive composition according to the present invention adds, to a food product containing cariogenic free sugars or derivatives thereof, also 0.001% to 0.01% by weight, preferably 0.002% to 0.003% by weight avocado oil as an agent for strengthening the dental support tissues.

Another subject matter of the present invention is a food product containing cariogenic free sugars or derivatives thereof, comprising a) an agent for reducing adhesion of dental biofilm, b) a mineralising agent based on silicon and/or based on calcium, and c) an antiseptic, all described previously, in the proportions described above for each of these compositions, namely:

a. about 79 to 99 parts by weight, preferably 90 to 96 parts by weight of xylitol as an agent for reducing adhesion of dental biofilm;

b. about 0.5 to 19 parts by weight, preferably 2.5 to 10 parts by weight of silica or a source of silica of plant origin such as horsetail as a remineralising agent;

c. about 0.2 to 2 parts by weight, preferably 0.4 to 1 part by weight of an antiseptic of plant origin such as sage,

said food product containing:

a. from 0.1% to 0.8% by weight, preferably 0.3% to 0.6% by weight xylitol as an agent for reducing the adhesion of dental biofilm;

b. from 0.005% to 0.02% by weight, preferably 0.01% to 0.02% by weight silica or a source of silica of plant origin, such as horsetail, as a remineralising agent;

c. from 0.001% to 0.01% by weight, preferably 0.002% to 0.003% by weight of an antiseptic of plant origin such as sage.

In another embodiment, the food product that is the subject matter of the present invention containing cariogenic free sugars or derivatives thereof may also comprise from 0.001% to 0.01% by weight, preferably 0.002% to 0.003% by weight of an agent strengthening the dental support tissues chosen from vegetable oils of avocado, evening primrose, borage or soya, or from animal oils of fatty fish containing omega 3 or 6.

For use thereof in a food product, the above percentages describe, for each component of the additive composition according to the invention, the final percentage of this component in the food product. Naturally this percentage reflects the mass of the component concerned for 100 g of solid food products, or for 100 g of liquid food product if applicable. According to the density of the liquid concerned, 100 g of this liquid can have a volume close to but different from 100 ml, which is the volume that occupies 100 g of water.

Preferably said food product contains, as free sugars, saccharose, glucose, fructose or any cariogenic polyol. It can in particular be chosen from sugar, confectionery, such as cocoa products or based on chocolate, including chocolate, chocolate bars or chocolate-based spreads, chewing gums, decorative products for cakes or icing; milk products, such as milky drinks, fermented or curdled milk products, condensed milks, milky desserts; sorbets or ice creams; products based on fruits, such as jams, jellies, marmalades, fruit-based spreads, candied fruits, pulps, purees, coatings based on fruits or coconut milk, desserts based on fruits; cereals and products based on cereals; bakery products, such as pancakes and cakes; sweeteners, including honey; preparations for babies, infants or young children, or intended for medical or diatetic use; coatings for medication; drinks such as fruit or vegetable juices, nectars, drinks based on flavoured water, sodas, coffee or substitutes, teas or infusions; health foods; food supplements; condiments such as mustard or ketchup; soups or broths; ready-cooked or semi-cooked dishes; frozen foods or composite foods.

The composition according to the present invention can in particular be incorporated in chocolate or more generally in a chocolate product, without causing any change to the flavour of the product.

Chocolate or chocolate product means any dark chocolate or milk chocolate or any product based on or containing such chocolates, that is to say composed of cocoa mass, cocoa butter, sugar, milk in the case of eponymous chocolates and optionally soya lethicin and/or other vegetable fats, and/or flavourings. Chocolate also means any white chocolate or any product based on or containing such chocolates, that is to say composed of cocoa butter, milk, sugar and optionally soya lethicin and/or other vegetable fats, and/or flavourings. This definition also includes the various forms that said chocolates or chocolate products may take, namely solid and liquid.

Said chocolate or chocolate product is characterised in that it contains:

a. from 0.1% to 0.8% by weight, preferably 0.3% to 0.6% by weight xylitol as an agent for reducing the adhesion of dental biofilm;

b. from 0.005% to 0.02% by weight, preferably 0.01% to 0.02% by weight silica or a source of silica of plant origin, such as horsetail, as a remineralising agent;

c. from 0.001% to 0.01% by weight, preferably 0.002% to 0.003% by weight of an antiseptic of plant origin such as sage.

In addition, said chocolate or chocolate product may contain from 0.001% to 0.01% by weight, preferably 0.002% to 0.003% by weight of an agent strengthening the dental support tissues.

Another object of the present invention is therefore a method of manufacturing chocolate making it possible to incorporate the previously described components of the food additive composition, characterised by the succession of following steps:

a. mixing the powders of xylitol as an agent for reducing adhesion of dental biofilm, of horsetail as a mineralising agent and of sage as an antiseptic;

b. grinding the mixture obtained at step a), preferably until a granulometry of less than or equal to 20 μm is obtained;

c. performing operations of kneading, refining, conching and tempering using cocoa paste, sugar and optionally cocoa butter and powdered milk;

d. incorporating the powdery mixture obtained at step b) in the chocolate paste obtained at the end of step c);

e. additionally, incorporating avocado oil in the chocolate paste obtained at the end of step c); and

f. casting or moulding the chocolate product obtained at step d).

In the steps of the above method, kneading means the step that is aimed at obtaining a homogeneous paste from sugar, cocoa paste and optionally cocoa butter and powdered milk. This step is performed in a mechanical pug mill. The paste obtained must have a particular texture, suited to the subsequent refining operation. It is possible to adjust this through choice of the granulometry of the sugar and also through the fat content.

Refining means the step that consists of rolling the paste obtained and discharging from the kneading between steel cylinders, so as to reduce the size of the particles to less than 25 microns. This operation transforms the initial paste into fine powder.

Conching means the step essential for modifying the flavour and improving the rheological characteristics of the chocolate. This step may last from several hours to several days. The refined powder is mixed hot, at around 75°-80° C. in the case of a dark chocolate and around 65° C. for white and milk chocolates.

Tempering means the step intended to crystallise the cocoa butter in a stable form. For this purpose, the chocolate paste is brought to a temperature of around 29° C.

Casting or moulding means the step for obtaining the desired shape or pattern for the chocolate. The latter is directly poured into moulds. This is moulding of a chocolate. Additional ingredients may be added at this time. The moulds and chocolate pass through a machine called a “tapoteuse” (tapper) that distributes the chocolate in the mould. Finally, it passes through a refrigerated tunnel that cools it instantaneously.

As explained previously, any sweetened food products with the composition according to the invention added, such as chocolate or a chocolate product, can be used as a product containing elements beneficial for dental health, in particular limiting the appearance of caries and strengthening the teeth, gums and dental bone.

### EXAMPLE 1

### Composition Added to Sugar

When it is intended to be incorporated in sugar, for example, the composition according to the present invention preferably contains xylitol as an agent for reducing adhesion of dental biofilm, horsetail as a source of silica and sage as an antiseptic.

| Ingredients | For 100 g of sugar |
|---|---|
| Xylitol | 100-800 mg (i.e. 0.1% |
| | to 0.8% by weight |
| Silica | 5-20 mg (i.e. 0.005% |
| | to 0.02% by weight) |
| Sage (Salvia | ≅2 mg (i.e. about |
| officinalis) | 0.002% by weight) |

### EXAMPLE 2

### Composition Added to Chocolate

When it is intended to be incorporated in chocolate or in a chocolate product, for example, the composition according to the present invention preferably contains xylitol as an agent for reducing adhesion of dental biofilm, horsetail as a source of silica as a mineralising agent, sage as an antiseptic and optionally avocado oil as an agent for strengthening the dental support tissues.

Preferably, the proportions are as follows:

| Ingredients | For 100 g of sugar |
|---|---|
| Xylitol | 100-800 mg (i.e. 0.1% |
| | to 0.8% by weight |
| Silica | 5-20 mg (i.e. 0.005% |
| | to 0.02% by weight) |
| Sage (Salvia | ≅2 mg (i.e. about |
| officinalis) | 0.002% by weight) |
| Avocado oil | ≅2 μl (i.e. about |
| | 0.002% by weight) |

For producing chocolate or chocolate product containing the food additive composition according to the invention and the proportions presented in the above table, for example, the xylitol and the horsetail and sage powders must be brought to a granulometry of 20 microns in order not to be perceptible in the mouth when they are mixed with the chocolate.

The following steps are then implemented in the following order: kneading, refining, conching, tempering, incorporation of the additive composition, moulding.

The steps of kneading, refining, conching, tempering and moulding are defined earlier in the description and are well known to persons skilled in the art specialising in the manufacture of chocolate and chocolate products. The present invention proposes to add a step of incorporating the food additive composition according to the present invention, after the tempering step, in the conventional chocolate manufacturing process.

The introduction of the additive composition must not take place before conching since the temperatures required for the latter (80° C. for saccharose) may impair the structure and efficacy of the various products. This is because at high temperature there may be polymerisation of the silicic acid of the horsetail and impairment of the active principles of the sage and avocado oil.

The xylitol must not be introduced with the saccharose (or the chosen sweetener) during the kneading phase since its conching temperature is lower. These increases in temperature may lead to a disorganisation of the crystalline states by melting of the xylitol crystals.

By way of example, 1 kg of dark chocolate was produced containing the food additive composition according to the present invention. For the kneading step, which is also the step of mixing the ingredients for obtaining a raw chocolate paste, 300 g of cocoa, 400 g of cocoa butter and 300 of sugar were mixed. The following step of grinding or refining was carried out by rolling the paste obtained at the discharge from the kneading between steel cylinders so as to reduce the size of the particles to less than 25 microns and obtain a much finer and more flexible paste. The conching step was carried out in rotary conches for 24 hours at 80° C. At this stage it is possible, where necessary, to add butter or lecithin according to the required fluidity. The tempering step was carried out by reducing the temperature of the mixture from about 50° C. to 18° C., in 10 minutes, under constant stirring. This temperature was kept constant for 10 minutes before reheating the mixture to achieve a temperature of 29° C. During this reheating step, the food additive composition, prepared moreover in the proportions given in the previous table and brought to a granulometry of less than 20 microns, was incorporated; 5 g of xylitol+200 mg of silica extracted from horsetail+20 mg of sage, mixed and brought to a granulometry of less than 25 microns were incorporated progressively and under constant stirring during the reheating phase described above, until there was homogeneous and total incorporation, maintaining the temperature constant at 29° C. At this same temperature, 20 μl of avocado oil was added while keeping the temperature constant at 29° C. The moulding step was carried out in order to obtain a chocolate in bars, using a vibrating machine for distributing the chocolate in the moulds and a refrigerated tunnel in order to cool it instantaneously. Also by way of example, 1 kg of milk chocolate was produced according to the same protocol using however the following proportions of ingredients during the kneading step: 100 g of cocoa+20 g of cocoa butter+500 of sugar+200 g of powdered milk.

### EXAMPLE 3

### Model for Studying the Reduction in Adhesion of a Dental Biofilm in Vitro

The study model used for measuring the reduction in adhesion of a dental biofilm in vitro is a proprietary technique of the company BioFilm Control, the BioFilm Ring Test®, protected by several patents FR 05/00427, FR 06/00578, FR 07/50891, FR 07/55344).

The test uses:

magnetisable microballs added to the culture medium for the microorganisms tested (S. mutans);

a block test where a 96-well plate is positioned precisely, to be tested over 96 magnets. The microballs are then attracted to the well bottom and form a spot, unless a biofilm immobilises them (no spot);

a reader scanning the 96-well plate before and after magnetisation;

BioFilm Elements® software analysing the image of the bottom of the wells of the 96-well plate in order to detect or not microball spots.

This technique is also described in Chavant et al., J. Microbiol. Meth 68 (2007) 605-612.

### Measuring the Reduction in Adhesion of a Biofilm of S. mutans in the Presence of Different Concentrations of Xylitol in Vitro

A biofilm of S. mutans is formed in a BHI medium in various wells of polystyrene microplates. The number of wells seeded depends on the experimental conditions tested. Each experimental condition is tested in triplicate, namely in three independent wells.

For preparing cultures in microplates, the S. mutans bacteria are spread over a Petri dish. From an isolated clone, an overnight preculturing (18 hours) is carried out in a Luria Broth (LB) medium at 37° C. and pH 6 or pH 7. The optical density (OD) of the preculture is then measured, adjusted to an OD of 1 and then diluted by a factor of 250 in a thermostatically controlled culture medium. A sufficient quantity of magnetic balls is added to the dilution (about 2 μl per well). Dilution of the preculture makes it possible to seed the various wells in the microplates intended for measuring the experimental conditions. The reference wells are not seeded.

After seeding of the wells by 200 μl of the diluted preculture, various culture conditions are tested in order to measure the effect of the xylitol on the adhesion of the biofilm of S. mutans. The effect of several xylitol concentrations between 0.05% and 5% w/v (g/100 ml), in particular 0.1; 0.2; 0.4; 0.8 and 5% w/v (g/100 ml), on the adhesion of the biofilm, is measured after times of between 2 and 48 hours of incubation at 37° C. (pH between 6 and 7) compared with the adhesion of the same biofilm in the absence of xylitol (0).

As described in Chavant et al., J. Microbiol. Meth 68 (2007) 605-612, measurement of the formation or not of microspots of magnetic balls at the bottom of each well reveals the presence or not of adherent bacteria and/or of a biofilm. Such adherent bacteria prevent the circulation of the magnetic balls in the direction of the magnets placed under each well and therefore the formation of microspots of balls at the bottom of these wells. On the other hand, a reduction in the adhesion of these bacteria by the xylitol enables the magnetic balls to circulate and microspots of balls to form.

Measurement of the more or less marked destructuring of the biofilm in the presence of xylitol may also be assessed by agitation of the biofilm formed as previously after an incubation time of between 2 and 48 hours in the presence of magnetic microballs. This agitation is standardised for each test microplate by means of a vortex. The shearing caused by the movement of the culture medium during the agitation allows remobilisation of the microballs related to the reduction in the resistance of the matrix of the biofilm and therefore a recording of the more or less marked destructuring of the biofilm by the release of microballs and viewing thereof or not, at the bottom of each well. In other words, these studies make it possible to quantify the resistance to agitation of the biofilm under the various experimental conditions.

These studies thus make it possible to determine in this in vitro model a minimum effective dose (MED) of xylitol for reducing adhesion of the biofilm of S. mutans.

### Measurement of the Reduction in Adhesion of a Biofilm of S. mutans at Different Concentrations of Xylitol in the Presence of Different Concentrations of Saccharose or Glucose in Vitro

The protocol for forming and seeding biofilm of S. mutans in the various wells of the polystyrene microplates is identical to that presented in the above paragraph.

The following experimental conditions make it possible to measure the influence of different concentrations of saccharose on the reduction in adhesion of the biofilm by the xylitol. The effect of several saccharose or glucose concentrations (between 0 and 20% w/v (g/100 ml), particularly 0, 5, 10, 15 and 20%) on the reduction in adhesion of the biofilm by a concentration of xylitol is measured after times of between 2 and 48 hours of incubation at 37° C. (pH between 6 and 7), compared with the reduction in adhesion of the same biofilm at the same concentration of xylitol in the absence of saccharose or glucose (0).

The effect of the saccharose or glucose is tested on the following concentrations of xylitol of between 0.05 and 5% w/v (g/100 ml), in particular 0.1; 0.2; 0.4; 0.8 and 5% w/v (g/100 ml), as described previously, and framing the M.E.D of xylitol reducing the adhesion of the biofilm.

These various respective doses of saccharose or glucose and xylitol make it possible to measure the reduction in adhesion of the biofilm of S. mutans for different ratios of “saccharose/xylitol” or “glucose/xylitol” concentrations.

As previously mentioned, the measurement of the more or less marked destructuring of the biofilm in the presence of xylitol and saccharose or glucose may also be assessed by agitation of the biofilm formed as above after an incubation time of between 2 and 48 hours in the presence of magnetic microballs. This agitation is standardised for each test microplate by means of a vortex. The shearing caused by the movement of the culture medium during the agitation allows a remobilisation of the microballs related to the reduction in the resistance of the matrix of the biofilm and therefore a recording of the more or less marked destructuring of the biofilm by the release of microballs and viewing thereof or not, at the bottom of each well. These studies make it possible to quantify the resistance to agitation of the biofilm under the various experimental conditions.

### Marking of a Biofilm with Acridine Orange and Simulation of a “Support Approach” Effect Increased Following the Reduction in Adhesion of the Biofilm by Xylitol in Vitro

By using fluorescent markers, in particular acridine orange, known for its qualities of marking cell structures and biofilms, traceable in confocal microscopy, it was possible to view the penetration of this fluorescent marker in the matrix of the biofilm under various experimental conditions and therefore to view under these conditions the destructuring of the biofilms synonymous with the reduction in adhesion thereof. The marking of the biofilms by acridine orange under the various experimental conditions will be proportional to this destructuring of the biofilms.

These experimental conditions include the use of several saccharose or glucose concentrations (between 0 and 20% w/v (g/100 ml), particularly 0, 5, 10, 15 and 20%) in the presence of xylitol at levels of between 0.05% and 5% w/v (g/100 ml), in particular 0.1; 0.2; 0.4; 0.8 and 5% w/v (g/100 ml), after incubation times of cells of between 2 and 48 hours incubation at 37° C. (pH between 6 and 7), compared with conditions in the absence of saccharose or glucose and xylitol.

The marking with acridine orange on cells incubated for a predetermined time, and then washed with phosphate buffer saline (PBS) will be carried out for 5 minutes, for example, by means of a solution of acridine orange at a conventional concentration of between 5 and 100 μg/ml. After marking and washing with PBS, the biofilms will be viewed by means of a confocal microscope (Zeiss LSM 510 Meta inverted microscope) and a quantification of the markings will be carried out by means of a suitable software.

By means of this approach, it is thus possible to view the destructuring of a biofilm by xylitol and thus demonstrate its greater permeability vis-à-vis potential plant active principles for approaching the support on which this biofilm is fixed. This approach therefore makes it possible thus to simulate a measurement of approach of a support by plant active principles through a biofilm of S. mutans having a reduction in adhesion.

### Supplementary Measurements in Vitro by Use of the Colouring Test with Crystal Violet

This test, derived from the test published in Musk et al. Chem. Biol. 12 (2005) 789-796, is a colorimetric test that makes it possible to quantify the adherent bacteria, in a biofilm for example, by measuring the crystal violet incorporated by them after successive steps of washing, marking, decolouring and dissolution of the coloured biofilm with acetic acid.

Thus, using a standardised protocol of rinsing biofilms set to grow under particular experimental conditions, in the presence of xylitol, saccharose and/or plant active principles for example, it is possible to compare the resistance to agitation of these biofilms and therefore the more or less marked destructuring of these under the various experimental conditions described in the above paragraph, using xylitol and saccharose or glucose.

Conventionally, this test requires a minimum incubation of bacteria for 48 hours in microplate wells. They are then washed by microplate washer water jet, making it possible to test the strength of the biofilms. They are then coloured by a crystal violet solution; by way of non-exclusive example, the marking can be effected by a 0.1% v/v solution in water for 45 minutes. The wells are then rinsed with water in order to remove the marking excess. The remaining bacteria coloured in the various wells are then dissolved by an acetic acid solution; by way of non-exclusive example, this solution may be a 33% v/v glacial acetic acid solution. After dissolution, the optical density of the solution recovered in each well is measured at 540 nm.

The values obtained are proportional to the number of adherent bacteria that resisted the rinsing. They therefore make it possible to quantify the resistance of the biofilms under different experimental conditions.

### Measurements of the Reduction in Adhesion of the Biofilm by Xylitol in the Presence of Plant Active Principles and Synergy thereof, in Vitro

The effect of the plant active principles coming from horsetail and sage on the reduction of the adhesion of the biofilm is measured by adding these under the experimental conditions of the various tests and approaches used previously. These various tests therefore make it possible to measure, in the presence thereof, apart from the reduction in adhesion of the biofilm, the resistance to agitation thereof. Concerning the marking of the biofilm with acridine orange, as explained in the corresponding paragraph, this test makes it possible to view and simulate a measurement of approach of a support by these plant active principles through a biofilm of S. mutans having a reduction in adhesion.

These various tests make it possible to measure the synergy between the plant active principles, coming in particular from horsetail and sage, and xylitol on the reduction in adhesion on a biofilm of S. mutans.

### REFERENCES

Bertrand et al. Les cahiers de l'ADF, No. 14-15, 2^{nd} quarter 2003.

Chavant et al., J. Microbiol. Meth 68 (2007) 605-612.

Engels-Deutsch et al. Les cahiers de l'ADF, No. 16-17, 3^{rd} quarter 2003,

Musk et al. Chem. Biol. 12 (2005) 789-796.

1. A food additive composition, comprising:
a) an agent for reducing the adhesion of dental biofilm,
b) a mineralising agent based on silicon and/or based on calcium,
c) an antiseptic.

2. The composition according to claim 1, wherein:
a) the agent for reducing the adhesion of dental biofilm is chosen from the group consisting of:
polyols such as xylitol, sorbitol, mannitol and erythritol, and hydrogenated disaccharides such as maltitol, lactitol and hydrogenated isomaltulose,
cranberry, coffee, chicory, inulin, tea, grapes, raisins, red wine,
b) the mineralising agent based on silicon is chosen from the group consisting of:
silica,
substances of plant origin extracted from plants including horsetail, lithothamnion, alfalfa, soya, nettle, bamboo or meadowsweet,
c) the antiseptic is a substance extracted from plants chosen from sage, echinacea, myrrh, propolis, ratanhia peruviana, calendula, yam, camomile, aloe vera, acerola, sanguinarine, lemon or grapefruit.

3. The composition according to claim 1, further comprising an agent strengthening the dental support tissues chosen from vegetable oils of avocado, evening primrose, borage or soya, or from animal oils of fatty fish containing omega 3 or 6.

4. The composition according to claim 1, further comprising xylitol as an agent for reducing the adhesion of dental biofilm, horsetail as a silica-based mineralising agent and sage as an antiseptic.

5. The composition according to claim 4, further comprising avocado oil as an agent strengthening the dental support tissues.

6. The composition according to claim 1, further comprising:
a. about 79 to 99 parts by weight, preferably 90 to 96 parts by weight of xylitol as an agent for reducing the adhesion of dental biofilm;
b. about 0.5 to 19 parts by weight, preferably 2.5 to 10 parts by weight of silica and a silica source of plant origin such as horsetail as a remineralising agent;
c. about 0.2 to 2 parts by weight, preferably 0.4 to 1 part by weight of an antiseptic of plant origin such as sage.

7. The composition according to claim 6, further comprising about 0.2 to 2 parts by weight, preferably 0.4 to 1 part by weight of an agent strengthening the dental support tissues.

8. The composition according to claim 1 for incorporating in a food product containing cariogenic free sugars or derivatives thereof, wherein the composition adds to said food product:
a. from 0.1% to 0.8% by weight, preferably 0.3% to 0.6% by weight xylitol as an agent for reducing the adhesion of dental biofilm;
b. from 0.005% to 0.02% by weight, preferably 0.01% to 0.02% by weight silica or a source of silica of plant origin, such as horsetail, as a remineralising agent;
c. from 0.001% to 0.01% by weight, preferably 0.002% to 0.003% by weight of an antiseptic of plant origin such as sage.

9. The composition according to claim 8, wherein the composition also adds to the food product containing cariogenic free sugars or derivatives thereof 0.001% to 0.01% by weight, preferably 0.02% to 0.003% by weight of avocado oil as an agent for strengthening the dental support tissues.

10. A food product containing cariogenic free sugars or derivatives thereof, wherein the food product comprises elements a), b) and c) of the composition as defined in claim 1.

11. The food product according to claim 10, further comprising 0.001% to 0.01% by weight, preferably 0.002% to 0.003% by weight of an agent strengthening the dental support tissues, chosen from plant oils of avocado, evening primrose, borage or soya, or from animal oils of fatty fish containing omega 3 or 6.

12. The food product according to claim 10, chosen from sugar, confectionery, such as cocoa products or based on chocolate, including chocolate, chocolate bars or chocolate-based spreads, chewing gums, decorative products for cakes or icing; milk products, such as milky drinks, fermented or curdled milk products, condensed milks, milky desserts; sorbets or ice creams; products based on fruits, such as jams, jellies, marmalades, fruit-based spreads, candied fruits, pulps, purees, coatings based on fruits or coconut milk, desserts based on fruits; cereals and products based on cereals; bakery products, such as pancakes and cakes; sweeteners, including honey; preparations for babies, infants or young children, or intended for medical or diatetic use; coatings for medication; drinks such as fruit or vegetable juices, nectars, drinks based on flavoured water, sodas, coffee or substitutes, teas or infusions; health foods; food supplements; condiments such as mustard or ketchup; soups and broths; ready-cooked or semi-cooked dishes; frozen foods or composite foods.

13. A chocolate or chocolate product, comprising:
a. from 0.1% to 0.8% by weight, preferably 0.3% to 0.6% by weight xylitol as an agent for reducing the adhesion of dental biofilm;
b. from 0.005% to 0.02% by weight, preferably 0.01% to 0.02% by weight silica or a source of silica of plant origin, such as horsetail, as a remineralising agent;
c. from 0.001% to 0.01% by weight, preferably 0.002% to 0.003% by weight of an antiseptic of plant origin such as sage.

14. The chocolate or chocolate product according to claim 13, further comprising from 0.001% to 0.01% by weight, preferably 0.002% to 0.003% by weight of avocado oil as an agent for reinforcing the dental support tissues.

15. A method of manufacturing chocolate according to claim 13, comprising the following succession of steps:
a. mixing the powders of xylitol, horsetail and sage;
b. grinding the mixture obtained at step a), preferably until a granulometry of less than or equal to 20 μm is obtained;
c. performing operations of kneading, refining, conching and tempering using cocoa paste, sugar and optionally cocoa butter and powdered milk;
d. incorporating the powdery mixture obtained at step b) in the chocolate paste obtained at the end of step c);
e. additionally, incorporating avocado oil in the chocolate paste obtained at the end of step c); and
f. casting or moulding the chocolate product obtained at step d).
